(19) United States
(12) Patent Application Publication
(10) Pub. No.: US 2015/0150677 A1
(43) Pub. Date: Jun. 4, 2015

(54) PROSTHETIC MECHANICAL HEART VALVE

(71) Applicant: Lakeway Innovations, LLC, Lakeway, TX (US)

(72) Inventors: Jonathan C. Stupka, Lakeway, TX (US); Michael R. Emken, Silver Creek, WA (US)

(21) Appl. No.: 14/404,937

(22) PCT Filed: May 31, 2013
(86) PCT No.: PCT/US2013/043638

§ 371 Date: Dec. 1, 2014

Related U.S. Application Data
(60) Provisional application No. 61/654,520, filed on Jun. 1, 2012.

(51) Int. Cl. A61F 2/24

(52) U.S. Cl. CPC A61F 2/2403

(57) ABSTRACT

A novel single occluder mechanical heart valve (1, 45) that exhibits reduced closing cavitation and potential blood-damaging hemolysis by utilizing guiderails (14, 54) to create a continuously shifting pivot axis to control hydraulic forces so as to minimize tangential velocity of the occluder (3, 53) at the instant of final closing. These forces are also used to effect a quick initial closing movement from the full open position. In the same way, other guiderails (12, 16, 52, 56) are employed to provide a quick opening response while guiding the occluder (3, 53) to its full open position where to two fairly equal flow channels are created.

This application claims priority from U.S. Provisional Application Ser. No. 61/654,520, filed Jun. 1, 2012, the disclosure of which is incorporated herein by reference.

### FIELD OF THE INVENTION

The invention relates to Prosthetic Mechanical Heart Valves and more particularly to single occluder valves.

### BACKGROUND OF THE INVENTION

Single occluder or mono-leaflet mechanical heart valves have an occluder that opens and closes in a defined way to alternately allow or block the flow of blood through, or out, of the heart. Mechanical valve design has progressed over the years with a sequence of improvements that have improved longevity, biocompatibility, hemodynamic performance, flushing of the pivot mechanism, reduction of turbulence, reduction of closing volume and resistance to cavitation.

While there have been inventions attempting to minimize high fluid shear and cavitation with clearance gaps between the housing and the leaflets or occluder in high velocity areas just prior to and at full close, little has been done with respect to pivot mechanisms to minimize the angular or tangential velocity of the leaflets or occluder arriving at the final closed position while still providing responsive closing movement during the first portion of the closing cycle. The likelihood of fluid shear in the blood to the degree that can lead to hemolysis increases with increasing leaflet tangential velocities just prior to full close.

Mechanical valves whose housings have opposing internal flats with internal recesses or external protrusions and have occluders (leaflets) with mating geometry generally have varying amounts of gaps in these regions. Current bi-leaflet mechanical valves also have varying amounts of gap between the housing interior wall and the leaflet peripheral edge near the vicinity of the housing interior wall in the full closed position. Such gaps are needed to promote flushing of the pivot mechanism, to eliminate potential for binding, to minimize cavitation and/or to accommodate manufacturing tolerances. Gaps in the pivot mechanism region are typically larger than those found along the housing interior wall to minimize the potential for stasis that can lead to thrombus formation. However, gaps sized for proper flushing of recessed pivot mechanisms can produce either blood damaging high velocity jets or excessive leak during backflow. It would be desirable to provide a uniform gap around the perimeter in the pivot area except where the occluder contacts the housing, as well as to provide prompt initiation of closing and opening yet minimize tangential velocity of an occluder at the instant of its final movement.

### SUMMARY OF THE INVENTION

This invention features a closing mechanism that continually shifts the pivot axis of the occluder from an initial position where the area of the occluder upon which reverse flow is trying to close the occluder is very substantially greater than the area where reverse flow is trying to keep it open. As the occluder pivots toward closure, the pivot axis continually shifts towards the center of the occluder where, at full closure, there is just slightly more area upon which flow is trying to close the occluder than area where flow is trying to open the occluder. The final pivot axis could be placed at the center of the surface area; however, with consideration to manufacturing tolerances and occluder stability, it is considered best to maintain a slight bias keeping the occluder in the full closed position.

Current mechanical valve designs generally have a predominately fixed pivot point on opening. This improved invention features an opening mechanism that also continually shifts the pivot axis of the occluder from an initial position where the area of the occluder upon which forward flow is trying to open the occluder is very substantially greater than the area where forward flow is trying to keep it closed. As the occluder pivots toward full open, the pivot axis continually shifts towards the center of the occluder where, at full open, there is just enough more area upon which flow is trying to open the occluder than area where flow is trying to close the occluder to allow the valve to become and remain fully open.

Another feature of this valve is that the central section or axial midsection of the valve body is preferably that of a right circular cylinder in the region of the guiderails; the closing and opening mechanisms require no opposing internal flats in the valve body with recesses (sockets) like most mechanical heart valve designs, along with the need for an occluder to have accompanying flat edge regions and protrusions along the peripheral edge. This improved valve employs minimal guiderails to effect desired occluder opening and closing motions. These minimal guiderails are formed in the cylindrical interior surface of the valve body so that, except for these rails, the geometric orifice area is as large as it can be for a given valve annulus diameter size minus the minimum amount of wall thickness required for structural integrity depending on materials used.

The peripheral surface of the occluder is essentially a section of a right circular cylinder. The amount of gap between the occluder and the housing in the full closed position is just enough to accommodate manufacturing fit-up tolerances and to minimize damaging levels of fluid shear and cavitation. The guiderails provide an open pivot mechanism design that essentially eliminates areas of potential stasis within the valve body. Tapered regions at the inflow and outflow valve body faces reduce flow vortices.

The preferred embodiment of this single occluder valve has the advantage of exemplifying a reduced hydraulic radius (minimal wetted surface area) and reduced obstruction to flow, resulting in lower pressure gradients. The occluder can have either a pair of concave and convex faces of substantially similar curvature and thus fairly uniform occluder thickness or a pair of flat, substantially parallel faces. The perimeter in either case has no protrusions or recesses and in the full closed position lies uniformly adjacent to the matching interior wall in the pivot area of the valve body. The occluder in the full open position is located such that its inflow face is relatively close to centerline of the valve body while still providing needed range of motion so as to yield two large flow channel areas.

In one particular aspect, the invention provides a prosthetic mechanical heart valve which comprises a generally annular housing having a central passageway, a single generally circular occluder shaped to close the central passageway through said housing, a first pair of generally diametrically opposed guiderails which cooperate with said occluder in its closing pivotal movement, a second pair of generally diametrically opposed guiderails which cooperate with said occluder in its opening pivotal movement, and a third pair of diametrically opposed guiderails which cooperate with said occluder later during its opening pivotal movement to prevent said occluder from traveling downstream, said first pair of guiderails having engaging surfaces which contact lateral regions of said occluder during its closing pivotal movement and create a pivot axis about which said occluder pivots to its closed position, which pivot axis shifts continuously so as to provide a quick and responsive closing pivotal movement during initial closing and then continuously slowing pivotal movement during the terminal closing cycle to minimize tangential velocity at the apexes of the occluder at final closing.

In another particular aspect, the invention provides a prosthetic mechanical heart valve which comprises a generally annular housing having a central passageway having an axially center region with an interior surface of a right circular cylinder, a single generally circular occluder positioned within said center region and shaped to close the central passageway through said housing, and a plurality of pairs of generally diametrically opposed guiderails which protrude from said interior surface of said center region and cooperate with said occluder in its opening and closing pivotal movements, said pairs of guiderails having a first set of engaging surfaces which contact lateral regions of said occluder during its closing pivotal movement and create a pivot axis about which said occluder pivots to its closed position, which pivot axis shifts continuously so as to provide a quick and responsive closing pivotal movement during initial closing and then continuously slowing pivotal movement during a terminal closing cycle to minimize tangential velocity at the apexes of the occluder at final closing.

### BRIEF DESCRIPTION OF THE DRAWINGS

FIG. 1 is a perspective view of a preferred embodiment of a single occluder valve with a mitral sewing cuff, showing the occluder in the full closed position and looking predominantly from the inflow end.

FIG. 2 is a perspective view of the valve of FIG. 1 looking predominantly from the outflow end.

FIG. 3 is a perspective view of the valve of FIG. 1 with an aortic sewing cuff showing the occluder in the full closed position and looking predominantly from the inflow end.

FIG. 4 is a perspective view of the valve of FIG. 3 looking predominantly at the outflow end.

FIG. 5 is a top inflow end view of the valve of FIG. 1.

FIG. 6 is a section view taken along line 6-6 of FIG. 5 looking at the guiderails.

FIG. 7 is a perspective view of the valve of FIG. 2 looking from the outflow end with the occluder and the sewing cuff removed.

FIG. 8 is a perspective view similar to FIG. 7 looking from the inflow end.

FIG. 9 is an enlarged top view similar to FIG. 5 with the occluder and sewing cuff removed.

FIG. 10 is a section view taken along line 10-10 of FIG. 9, where the location of the valve axis is shown.

FIG. 11 is a section view showing the valve of FIG. 10, with the occluder in an early closing position, oriented about 5 degrees off the valve axis. To improve clarity, cross sectioning is omitted in FIGS. 11-26.

FIG. 12 is an enlarged detailed view of the indicated region of FIG. 11 showing the contact point which defines the pivot axis between the occluder and the guiderail. Also shown are a series of points marking pivot axes which constitute the percentage splits of surface area where backflow is closing the valve versus trying to open the valve.

FIG. 13 is a section view like FIG. 11 with the occluder having moved to a closing position about 30 degrees off valve axis.

FIG. 14 is an enlarged detailed view of the region indicated in FIG. 13. showing the contact point which defines the pivot axis between the occluder and the guiderail at that time.

FIG. 15 is a section view like FIG. 13 with the occluder in a closing position about 60 degrees off the valve axis.

FIG. 16 is an enlarged detailed view of the region indicated in FIG. 15 showing the contact point which defines the pivot axis between the occluder and the guiderail.

FIG. 17 is a section view like FIG. 15 with the occluder shown in the full closed position.

FIG. 18 is an enlarged detailed view of the region indicated in FIG. 17 showing the final contact point which defines the pivot axis between the occluder and the guiderail.

FIG. 19 is a section view like FIG. 17 with the occluder positioned at the start of the opening cycle where it has just shifted downstream to contact the opening guiderail.

FIG. 20 is an enlarged detailed view of the region indicated in FIG. 19 showing the contact point which defines the pivot axis between the occluder and the guiderail.
Also shown are a series of points marking pivot axes which constitute the split of surface area where forward flow is opening the valve versus that where it is trying to close the valve. Pivot axis values are provided as the percent of overall surface area of the occluder upon which forward flow is opening the valve.

FIG. 21 is a section view like FIG. 19 with the occluder in an opening position about 60 degrees off valve axis.

FIG. 22 is an enlarged detailed view of the region indicated in FIG. 21 showing the contact point which defines the pivot axis between the occluder and the guiderail.

FIG. 23 is a section view like FIG. 21 with the occluder in an opening position about 30 degrees off the valve axis.

FIG. 24 is an enlarged detailed view of the region indicated in FIG. 23 showing the contact point which defines the pivot axis between the occluder and the guiderail.

FIG. 25 is a section view like FIG. 23 with the occluder in an open position about 5 degrees off the valve axis.

FIG. 26 is an enlarged detailed view of the region indicated in FIG. 25 showing the contact point which defines the pivot axis between the occluder and the guiderail.

FIG. 27 is a perspective view of a valve having housing featuring an alternative guiderail design and a flat occluder that accomplishes essentially the same continuously moving pivot action as the preferred embodiment previously shown. This view looking predominantly at the valve inflow end shows the occluder in the open position about 5 degrees off the pivot axis.

FIG. 28 shows the valve of FIG. 27 with the occluder in the full closed position.

FIG. 29 is a perspective view of the valve of FIG. 28 looking predominately at the outflow end of the valve.

FIG. 30 is a perspective view of the valve of FIG. 27 looking predominately at the outflow end of the valve.

FIG. 31 is a top inflow end view of the valve of FIG. 28 with the occluder in the closed position.

FIG. 32 is a section view taken along line 32-32 of FIG. 31.

### DETAILED DESCRIPTION OF THE PREFERRED EMBODIMENTS

The following specification taken in conjunction with the drawings set forth the preferred embodiment for this invention although it should be understood that modifications can be accomplished within the scope of the present invention.

Shown in the drawings is a preferred embodiment which is a mono-leaflet or single occluder valve 1 incorporating various novel features. The valve 1 comprises a valve body or housing 2, an occluder 3 and either a mitral sewing cuff 4 or an aortic sewing cuff 6. Both valves are shown in the closed position.

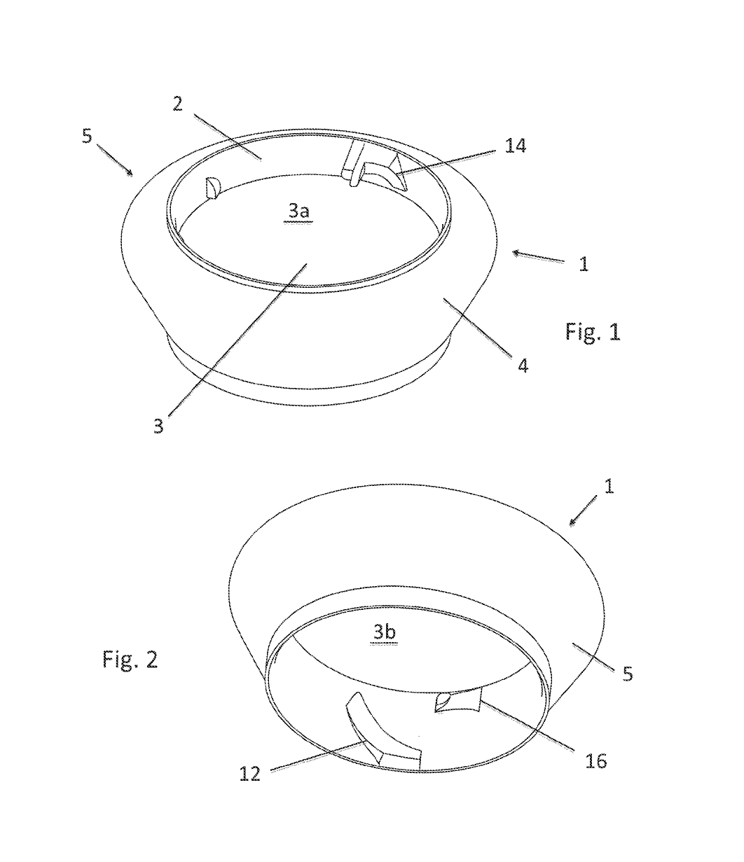

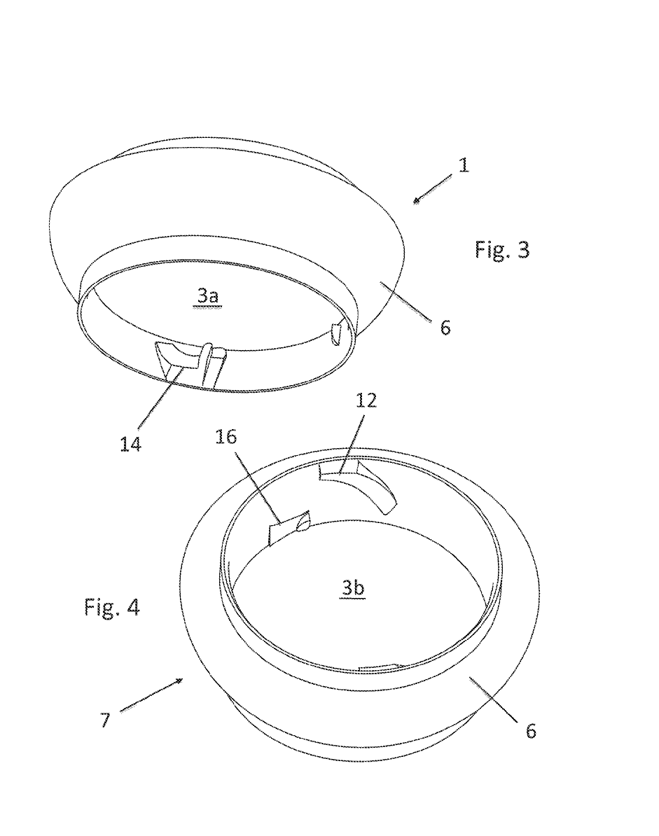

FIGS. 1 and 2 show the valve 1 as part of a mitral valve assembly 5 with a mitral sewing cuff 4, while FIGS. 3 and 4 show the same valve 1 as a part of an aortic valve assembly 7 with an aortic sewing cuff 6. With different sizes of housings and occluders with and variations on these two sewing cuffs, this valve design can be used to replace any of the four native human heart valves for all but the extremes of the patient population.

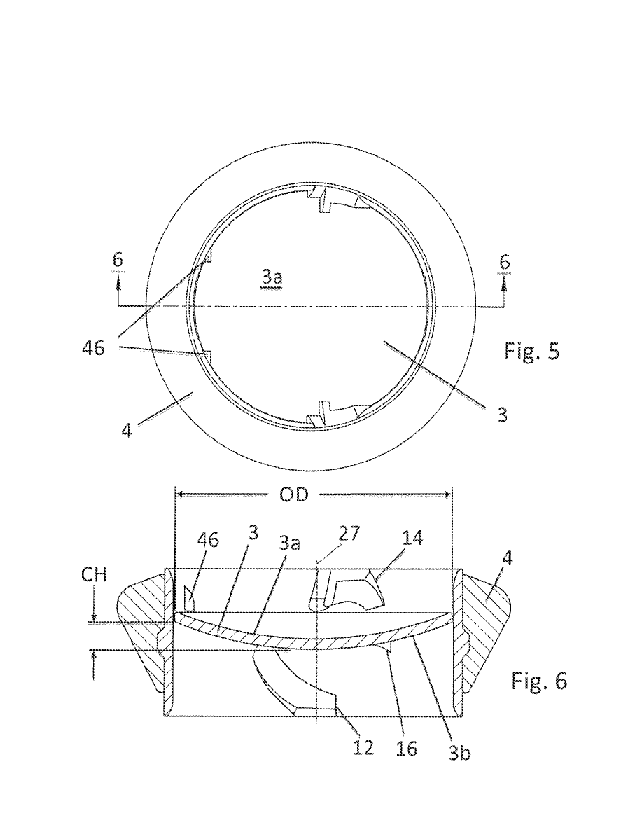

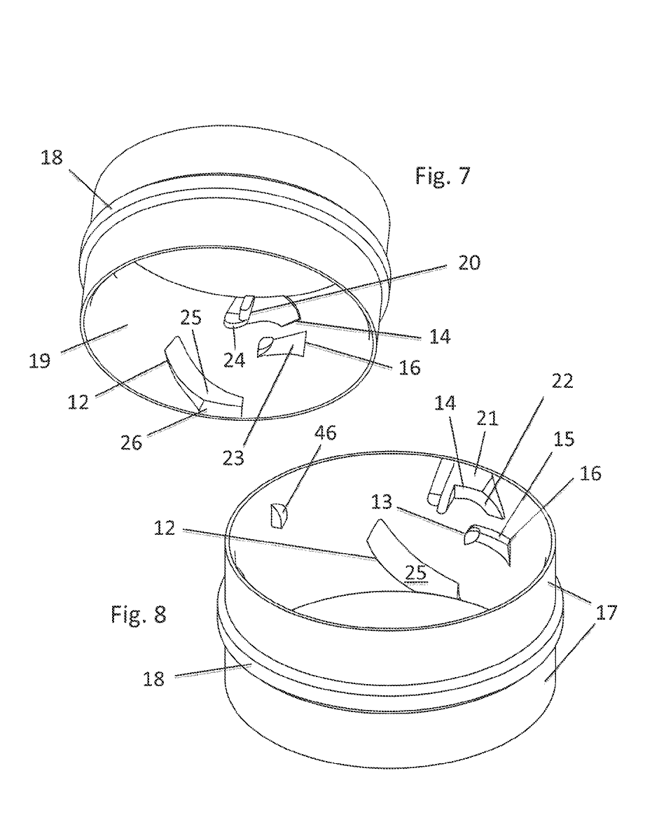

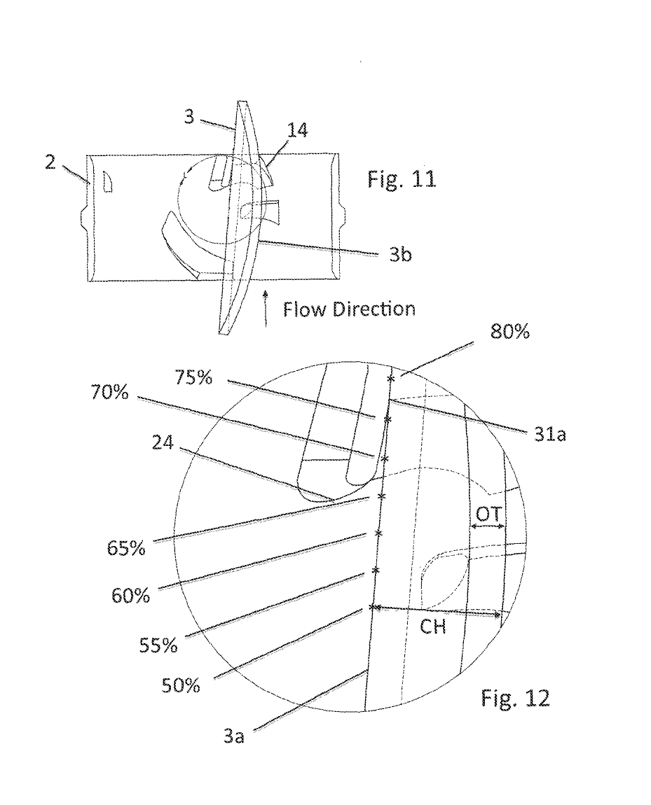

As seen in FIGS. 7 and 8, the housing has a generally cylindrical outer surface 17 that is interrupted by a sewing cuff flange 18 that is used both to increase the rigidity and provide a positive means of attaching a rotatable sewing cuff to the housing as is commonly known in the industry. At least the midsection of the interior surface 19 of the housing (FIG. 7) is predominately a right circular cylindrical surface; there are no interior flats, such as are common to many of today's commercial prosthetic mechanical valve designs. The height or length L of the housing 2 (FIG. 10) is intermediate between high profile valves and low profile prosthetic valves currently commercially available. It may be termed a medium height valve, which allows the valve to be placed interannularly in the heart annulus with a supra-annular sewing cuff that provides superior internal orifice area relative to a heart's annular root while also minimizing the potential for tissue or pannus ingrowth that can potentially impede desired occluder motion. The periphery of the occluder 3 (FIGS. 1 & 5) is fairly described as being generally circular or a cylindrical projection relative to the valve axis 27 as shown in FIGS. 5 & 6. The occluder 3 has an inflow face 3a that is generally concave and an outflow face 3b that is generally convex. The amounts of concavity and convexity of each, i.e. the arcuate radii, are such that they produce a generally constant occluder thickness OT (FIG. 12). The chord height CH of the convex surface 3b can range between about 2.5% and 15% relative to the outer diameter of the occluder OD. Preferably, it is about 10%.

There will generally be about 2 mm axial length of exposed housing outside surface extending beyond the sewing cuff that will seat in the heart annulus for both a mitral valve assembly 5 (FIG. 1) and an aortic valve assembly 7 (FIG. 3). The housing 2 is preferably formed with radially tapered inlet 8 (FIG. 10) and outlet 10 leading to and from its interior surface 19. As a result, the inlet edge 9 and outlet edge 11 are reduced in thickness, minimizing flow vortices during both forward flow and reverse flow through the valve. The tapered inlet 8 (FIG. 10) and outlet 10 have a thickness T2 (FIG. 10) of about 60% of the thickness T1 of the housing, i.e., the distance between the housing midsection interior surface 19 and outer surface 17, measured at the valve inlet and outlet edges 9 and 11. The inflow surface 3a is the one which faces incoming bloodstream that passes through the valve as the valve is opening during normal operation; in other words, it faces the inlet of the valve 1 in the closed position.

Preferred material choice for the housing 2 and occluder 3 is pyrolytic carbon (alloyed or unalloyed) commonly used for current commercial mechanical valves in the form of a pyrolytic carbon-coated graphite substrate. This time tested and proven material has outstanding biocompatibility, fatigue resistance and strength for mechanical heart valve applications. Housing material could also be a titanium alloy, while an alternative material for the occluder 3 could be a polymer, such as an acetal homopolymer (commonly known as Delrin®) or a polyether ether ketone (PEEK). Preferred material for the sewing cuffs 4 and 6 would either be a woven PTFE or Dacron. Sewing cuff retention rings (not shown in the drawings) used to affix the sewing cuff to the housing would be a titanium alloy.

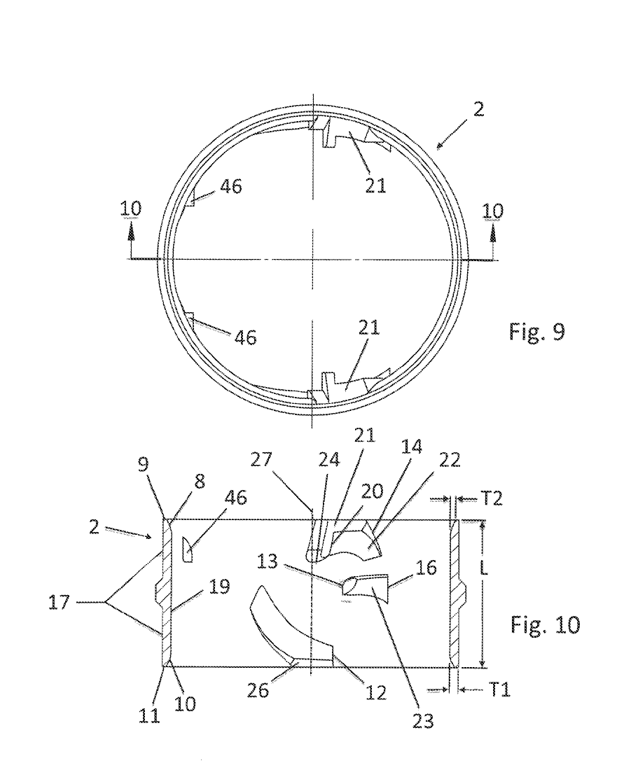

The preferred embodiment of the valve body or housing 2 has no internal opposed flats with recessed sockets or protruding pivots along with mating occluder geometry. Nor does it have struts, posts or seating lip features commonly used in mono-occluder valves. Rather, it employs a plurality of pairs of guiderails 12, 14 and 16 of minimal thickness that are designed to provide desired occluder motion with sufficient capture to eliminate the possibility of escape, while the full closed and open contact areas between the components are sized to maintain stresses within acceptable levels. As seen in FIGS. 6, 7 and 8, for example, three pairs of generally diametrically opposite guiderails are employed. They are functionally referred to as opening guiderails 12, closing guiderails 14 and initial opening guiderails 16. Guiderails 16 have upstream facing curved surfaces 15 and arcuate surfaces 13. For consistency, FIGS. 5 to 26 are all oriented similar to the mitral valve assembly 5 seen in FIG. 1.

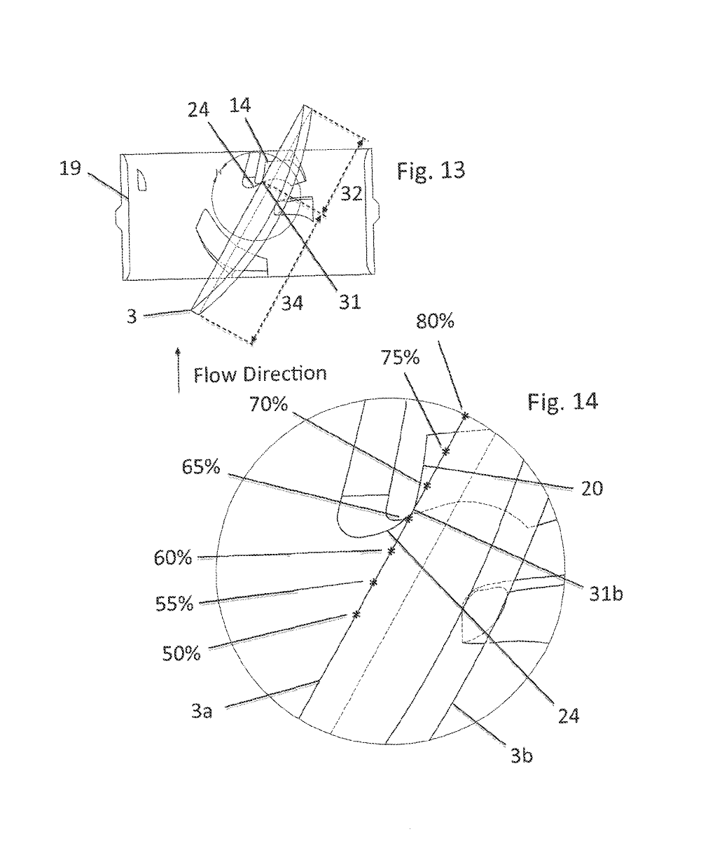

During closing of the illustrated valve 1, the points of contact between the occluder 3 and the opposite closing guiderails 14, i.e., the pivot-axis-determining points between the occluder 3 and housing 2, determine the effective amount of surface area 34 (see FIG. 13) of the occluder where backflow or reverse flow fluid pressure is trying to close the valve versus the effective surface area 32 where such pressure is attempting to maintain the valve open at that particular instant. Currently commercially available mechanical heart valves have a predominately fixed pivot point (as viewed in a plane perpendicular to the pivot axis) at least from an occluder orientation about 15 degrees from the valve axis into the closing cycle to full closed, and this fixed pivot axis is located where there is a fairly high ratio of surface area upon which backflow (see Flow Direction arrow in FIG. 13) is trying to close as opposed to that which would maintain the valve open. This causes an occluder to continue to accelerate towards full closed position. In contrast, the illustrated embodiment of the valve 1, which incorporates a closing mechanism without flats and associated pivot sockets or protrusions, has a high ratio of surface area where backflow is initially acting to close rather than open the valve thereby providing the desired initial responsiveness. However, as the occluder 3 moves towards its full closed position, the ratio of surface area where backflow is acting to close the valve rather than maintain it open continually decreases; this minimizes occluder velocity as the occluder approaches the valve body interior surface 19. This ratio of surface area continuously changes, i.e. decreases, throughout closing until it reaches a point where, at the instant of reaching full closure, there is only a slightly greater surface area where backflow is acting to close rather than open the valve.

To maximize efficiency, it is important for a valve to close as quickly as possible to minimize backflow past the occluder which continues until the valve occluder reaches its fully closed position. The amount of backflow passing through the valve during closure is often called closing volume. Closing volume plus leakage through the valve during its full closed state is often referred to as regurgitation. Regurgitation reduces heart pumping efficiency as it represents a volume of blood that needs to be “pumped twice”. As important as it is to increase efficiency, it is as, or perhaps more, important to minimize and ultimately eliminate high levels of turbulence, fluid shear and cavitation in the blood that can lead to hemolysis (blood damage). While mechanical heart valves have the advantage of longevity over tissue valves, their downside, up until now, has been the generation of some hemolysis, often to levels requiring a lifelong regimen of anti-coagulation for most patients, especially those of non-Asian descent. Whereas recent mechanical valve designs have generally focused on reducing forward flow turbulence and backflow cavitation at and beyond full close position, recent work has revealed that flow in the region between the occluder and the housing in the final stages of closure can give rise to hemolysis-generating fluid flows.

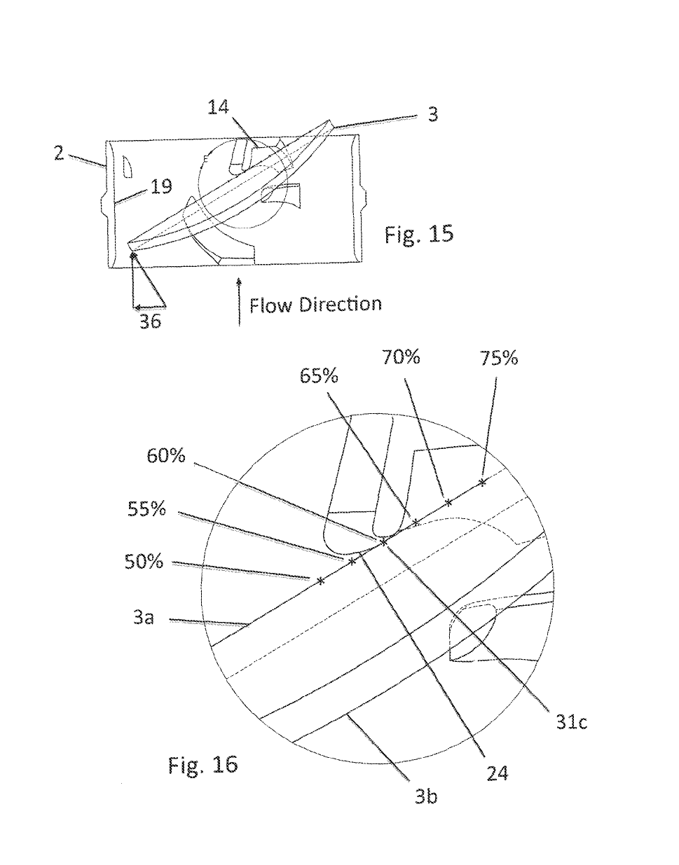

An important component influencing fluid flow during this segment of valve closure is the velocity of the occluder apexes relative to the housing internal wall. The component of concern is the tangential component 36 of occluder apex velocity depicted in FIG. 15. The apexes should be understood to be the two diametrically opposed tips of the occluder 3 that approach the housing interior wall surface at the time of full closing. Angular velocity due to the occluder pivoting about an axis has two components, tangential and axial. Angular velocity is the hypotenuse of the triangle depicted, and the two components, axial and tangential velocities, form the sides of the resulting right triangle. The focus of the tangential component 36 is the motion at the occluder apex as seen at the housing rectilinear wall surface, and this seems to have the most influence on fluid shear.

Previous valve designs have a predominantly fixed pivot axis of the occluder during most of the closing cycle and particularly during the final stage. This produces a predominantly fixed ratio of backflow trying to close the valve rather than open it. Some bi-leaflet valve designs have employed a shifting pivot axis or camming effect to initially positively shift the leaflets from fully open positions where they are parallel to flow, but they then employ a substantially fixed pivot for the remainder of the closing cycle (see U.S. Pat. No. 5,641,324). As a result, such shifting mechanism is in effect for only the first about 10° of the closing cycle and only for a minor distance of movement along the surface of leaflet ears that are received within cavities in the housing.

The design of valve 1 creates a continuously shifting pivot axis throughout the substantially complete closing and opening cycles; this results in a lowering of both angular and tangential velocity near the end of each cycle. The pivot axis is located so that forces on the outflow surface 3b are highly biased in the initial portion of the closing cycle to quickly accelerate the occluder closing motion, thereby minimizing closing volume; however, there is a dramatic and significant shift of the pivot axis to a near neutral location where the ratio of the amount of backflow closing the valve is close to the same as the amount of backflow trying to maintain the valve open during the last segment of the closing cycle, which minimizes tangential velocity and hemolysis. It is believed important that the design be such as to rapidly maximize occluder velocity at its initial closing movement but then truly minimize tangential velocity in the moments before reaching the full closed position in order to minimize hemolysis. Moreover, the housing interior wall in the regions which juxtapose with the occluder apexes may be only generally circular, e.g. the shape of a tabulated cylinder, to further minimize hemolysis. In the preferred embodiment, a pair of occluder stops 46 are provided (see FIGS. 5 & 6) which positively prevent the occluder from possibly over-rotating once the occluder 3 reaches its fully closed position.

During closing movement, there are two zones of contact between lateral regions of the occluder 3 and each of the two closing guiderails 14. One zone is between the peripheral edge at the inflow surface 3a of the occluder, generally at its slightly rounded edge, and the slightly curved surface 20 of the guiderail 14 (see FIG. 10), which contact is present during the first part of the closing cycle, as seen in FIGS. 11 and 12 showing initial closing movement, and the point of which shifts therealong until it reaches the adjacent arcuate surface 24 during the latter portion of the closing cycle. The instantaneous pivot axis (FIG. 13), which is defined by contact between the occluder 3 and the guiderail surface 20 of the housing continuously shifts along the curved surfaces 20 and 24 where it is labeled with the reference numbers 31, 31a, 31b, 31c and 31d (see FIGS. 7, 12, 14, 16 and 18). The geometry of guiderail surface 24 is designed to ensure stresses of the valve, in its full closed state, are well within the strength limits of the material used.

The second zone of contact with the occluder 3 occurs along contoured surface 22 of the guiderail 14. This zone of contact provides capture needed to ensure the occluder 3 does not escape upstream during backflow. Contact in this zone, along with the contact in the zone where the continuously shifting pivot axis 31 is being defined, controls the amount of surface area effectively closing the valve, versus the amount of surface area trying to open the valve, as the occluder 3 moves through its closing cycle. The upstream-facing surface 21 of guiderail 14 is contoured to minimize turbulence and stasis as the valve opens and closes; it does not make contact with the occluder 3. Surface 22 of guiderail 14 is also contoured to minimize flow disturbances and stasis while still providing needed occluder closing functionality.

At the moment of initial closing motion, when the occluder surface 3a makes initial contact 31a with the guiderail 14 (FIG. 12), the amount of surface area of the occluder where backflow is attempting to close the valve will be at least about 65% and preferably at least about 75% of the total outflow face area 3b. This amount assures prompt acceleration of occluder movement. However, the design is such that this greater percentage of surface area where backflow is closing the valve continually decreases throughout substantially the entire closing cycle following its initial acceleration until, upon reaching the point of full close 31d (FIG. 18), there is no more than about 53%, and preferably no more than about 51% of the total outflow face 3b surface area that is closing the valve. Although the pivot axis could be neutral, where the surface area closing the valve is about the same as the surface area opening the valve, it is felt best to have a slight bias in favor of the amount of surface area closing the valve.

FIGS. 11 & 12 show the occluder 3 positioned in the initial stages of the closing cycle where the occluder has shifted to contact the guiderails 14. The surface 3a of the occluder makes contact with the guiderail 14 at about the point 31a, which defines the pivot axis at this moment. It can be seen from the series of points marked along the surface 3a that the initial pivot axis 31a lies at about the line across the occluder surface where there is about 77% of the total outflow face 3b surface area upon which backflow is acting to close the valve. FIGS. 13 & 14 show the occluder 3 having moved to a position where it is now oriented about 30 degrees off the valve axis 27 (FIG. 10). At this instant in closing movement, occluder surface 3a contacts guiderail surface 20 at point 31b. At about this position, contact of surface 3a with guiderail 14 is shifting to about the end of the slightly curved surface 20 of each guiderail close to where it transitions to the arcuate section 24. In FIG. 14, the pivot axis 31b lies close to a line across the occluder surface where there is about 66% of the total outflow face 3b surface area upon which backflow is acting to close the valve.

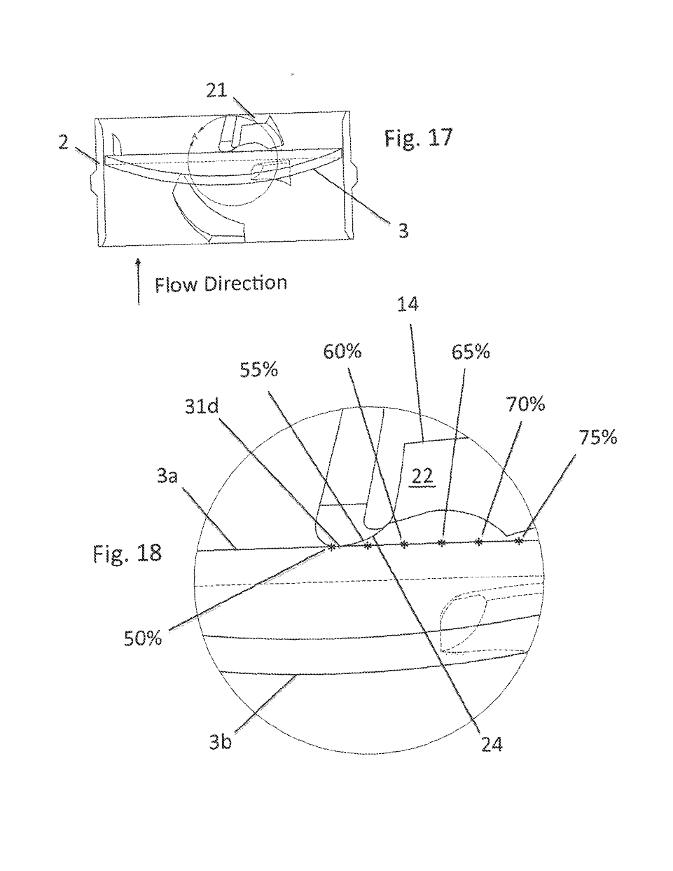

FIGS. 15 & 16 show the occluder 3 having moved to a position about 60 degrees off the valve axis 27. Here, occluder surface 3a contact with the guiderails 14 is now with arcuate surface 24 of each guiderail, defining a pivot axis 31c where there is about 60% of the total outflow face 3b surface area upon which backflow is acting to close the valve. FIGS. 17 & 18 show the occluder 3 in the full closed position there is about 52% or less surface area acting to close the valve. As shown, occluder surface 3a contact 31d with the arcuate end surface 24 of the guiderail is close to the point where there is only about 51% of the total outflow face 3b surface area upon which backflow is acting to close the valve.

A similar arrangement is used to effect valve opening; opening guiderails 12 and initial opening guiderail 16 (FIG. 7) are also designed to provide a continually shifting pivot axis throughout the opening cycle so that initial forward flow, downward through the valve body 2 as depicted in the drawings, acts on a large ratio of the surface area 3a that opens the valve, rather than closes the valve. As the occluder 3 moves towards its full open position, the ratio of surface area where forward flow is acting to open, rather than close, the valve continually decreases, and as a result in the full open state, there is only a small excess amount of surface area where forward flow is acting to open rather than close the valve occluder 3, which assures the valve will stay fully open during peak forward flow. The benefit of a continually shifting pivot axis during valve opening is the achievement of a responsive quick opening while minimizing occluder velocity when the occluder reaches near full open position. The positioning of the guiderails and the occluder in the full open state is such to create two resulting flow channels that are fairly similar in size while still ensuring desired overall valve function.

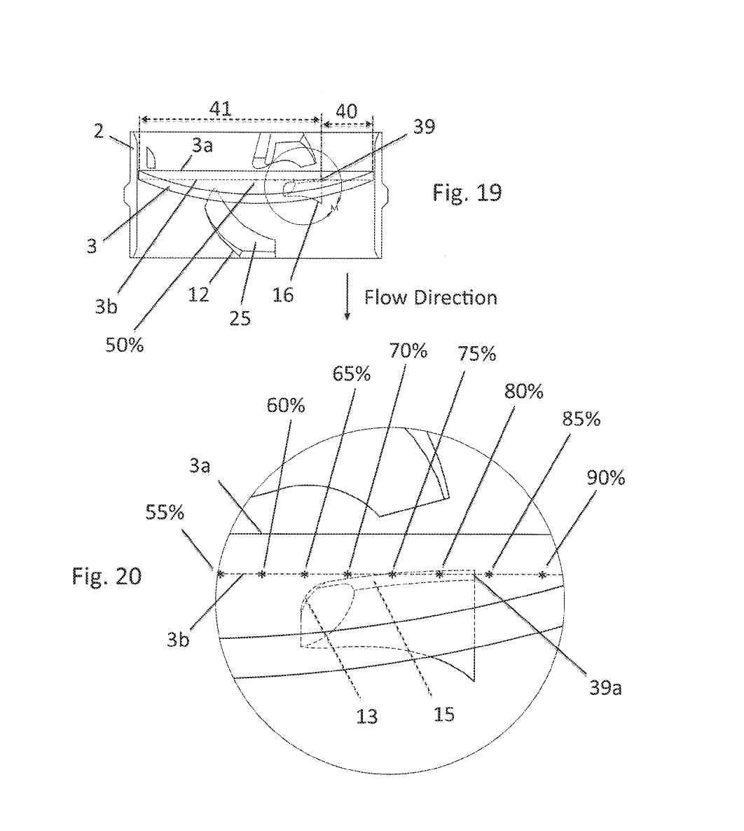

At the start of opening movement (FIG. 19), the occluder 3 of the preferred embodiment is initially displaced downstream so the outflow surface 3b, generally at its slightly rounded edge, contacts the upstream-facing curved surface 15 of the guiderail 16. As opening movement progresses, the contact between occluder 3 and housing 2 moves serially points along 39a-39d in FIGS. 19-26; these contact points define the opening pivot axis between the occluder 3 and housing 2, which determines the amount of total occluder inflow surface area 3a where blood flow is trying to open the valve (see region 41 in FIG. 19) versus that trying to close the valve (see region 40).

There are two zones where contact occurs between the occluder 3 and each set of opposite opening guiderails 12 and 16. The first zone comprises the continuously shifting pivot points 39 which advance along the curved surface 15 (FIG. 20) of the guiderail 16 during the initial portion of the opening cycle and transition to the adjacent arcuate surface 13 as the occluder 3 moves towards the end of the opening cycle. The geometry of guiderail surfaces is designed to ensure stresses of the valve in its full open state are well within the strength limits of the material used. The second zone where contact occurs is located along a curved surface 25 of the opening guiderail 12 (FIG. 19). This second zone of contact with the occluder 3 provides capture needed to ensure the occluder does not escape during forward flow. This second zone along with the first zone assists in determining pivot points that define the continuously shifting pivot axis that determines the amount of surface area opening the valve on one side of the pivot axis versus the surface area tending to close the valve as the occluder 3 moves through the opening cycle. Downstream facing surface 23 of guiderail 16 and surface 26 (FIG. 7) of opening guiderail 12 are contoured to minimize turbulence and stasis as the occluder opens and closes. Likewise, operative surfaces 15 of the guiderail 16 and 25 of guiderail 12 are contoured to minimize flow disturbances and stasis while still providing needed occluder opening functionality for the shifting pivot axis.

On initial opening, the amount of surface area 3a on one side of the pivot axis 39 where forward flow is opening the valve is at least about 65%, preferably at least 75% and most preferably at least about 80% of the total surface area. The percentage of surface area where forward flow is opening the valve continually decreases until, at the point of full open, there is no more surface area trying to open the valve than required to assure the valve will remain fully open during peak forward flow. For the preferred embodiment, this may be about 65%. This arrangement creates a prompt initial valve opening response which accelerates occluder pivotal movement while also allowing the occluder 3, in its full open state, to be oriented relative, to the housing, such that it creates two fairly equal flow channels without requiring an overly high housing height.

FIGS. 19 & 20 show the occluder 3 positioned at the start of the opening cycle, having been just displaced downstream from its full closed position. Occluder contact with the curved surface 15 of the guiderail 16 defines a pivot axis 39a that is positioned close to a line across the occluder 3 where there is about 83% of the total surface area 3a upon which forward blood flow is opening the valve.

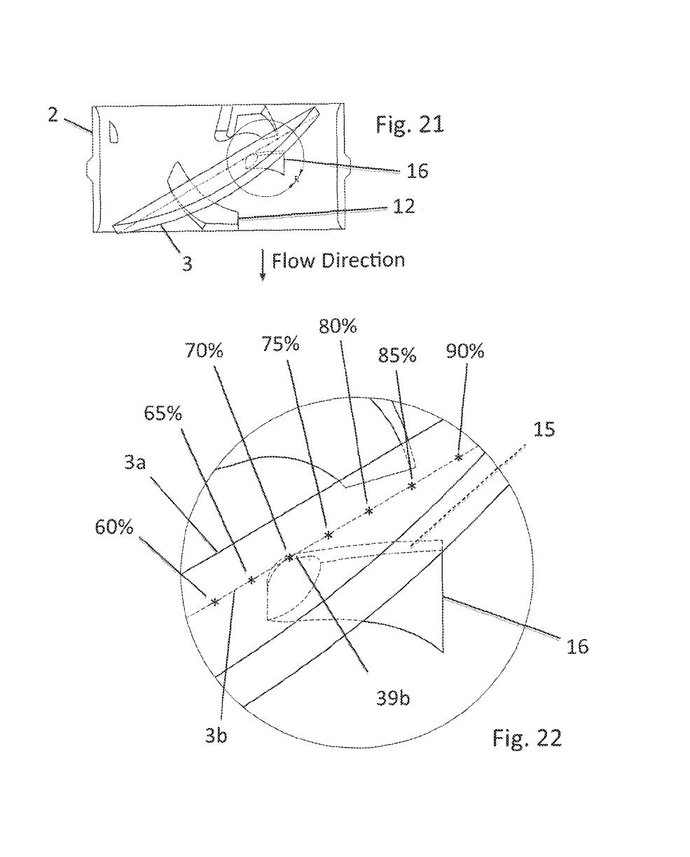

FIGS. 21 & 22 show the occluder 3 having pivoted to a position about 60 degrees off valve axis 27. Here, occluder contact is with each guiderail at the point 39b on the curved surface 15 defining a pivot axis 39b that is close to a line across the occluder where forward flow upon about 71% of the total surface area 3a is opening the valve.

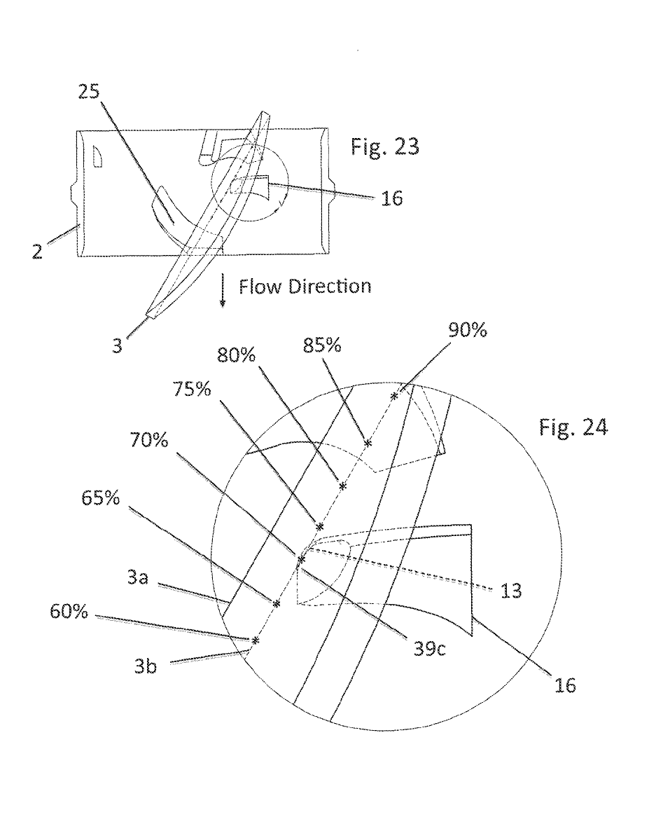

FIGS. 23 & 24 show the occluder 3 positioned at an orientation about 30 degrees off the valve axis 27. Here, occluder contact with each guiderail is at a point 39c defining a pivot axis that is close to a line across the occluder surface 3b where there is about 69% of the total surface area 3a upon which forward flow is opening the valve.

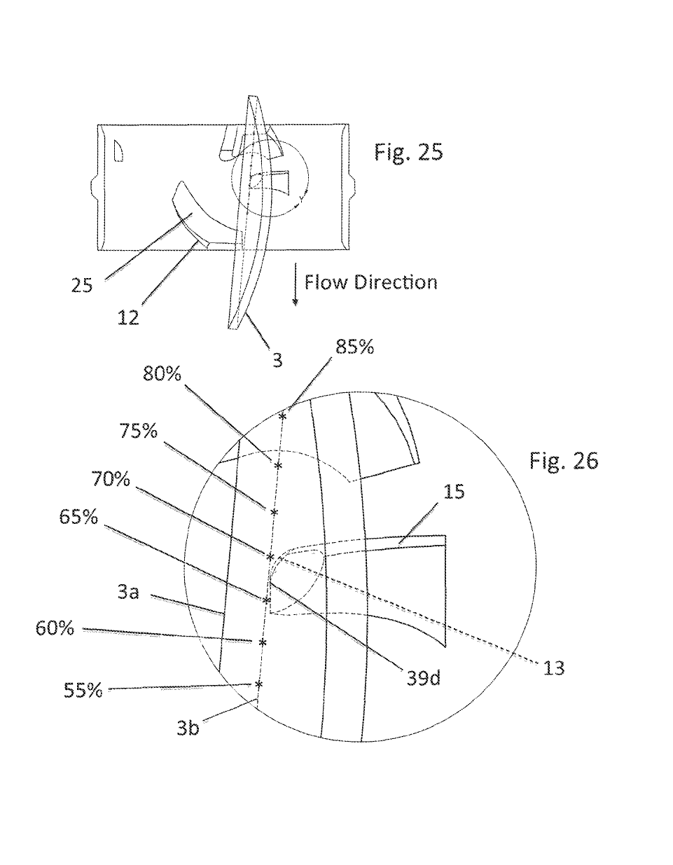

FIGS. 25 & 26 show the occluder 3 in the final stages of opening. Here, occluder contact with the arcuate surface 13 of the guiderail is at the point 39d which defines a pivot axis that is close to a line across the occluder surface 3b where there is about 67% of the total surface area 3a where forward flow is opening the valve. Throughout most of the opening cycle, lateral regions of occluder 3 are also in contact with the curved surfaces 25 of the guiderails 12 which prevent it from traveling downstream. In the final open position, the percentage trying to open the occluder 3 will generally not be less than about 53%, and will preferably be at least about 60%.

In addition, the preferred embodiment of valve 1 is designed to have a uniform clearance between the housing and the occluder perimeter within the pivot actuation region or in the vicinity of the guiderails. This clearance is not more than required to prevent binding or sticking and to accommodate manufacturing tolerances

Many valve designs employ internal opposing flats that blend into the housing internal diameter containing recesses and/or protrusions to control and guide occluders or leaflets through their opening and closing cycles. These designs require relatively large amounts of clearance between occluder/leaflet geometry and the housing recesses or protrusions than in the flats to promote more flow during full close in attempts to minimize areas of stasis. Other valves, which do not have such flat areas, generally require seating lips, struts or posts to promote uniformity of clearance between the housing and the occluder(s).

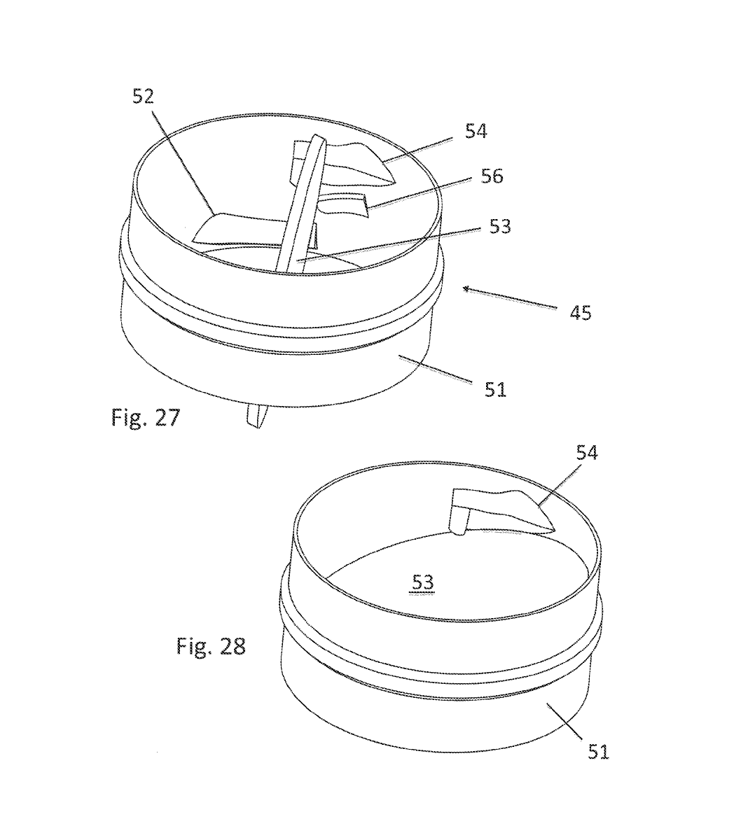

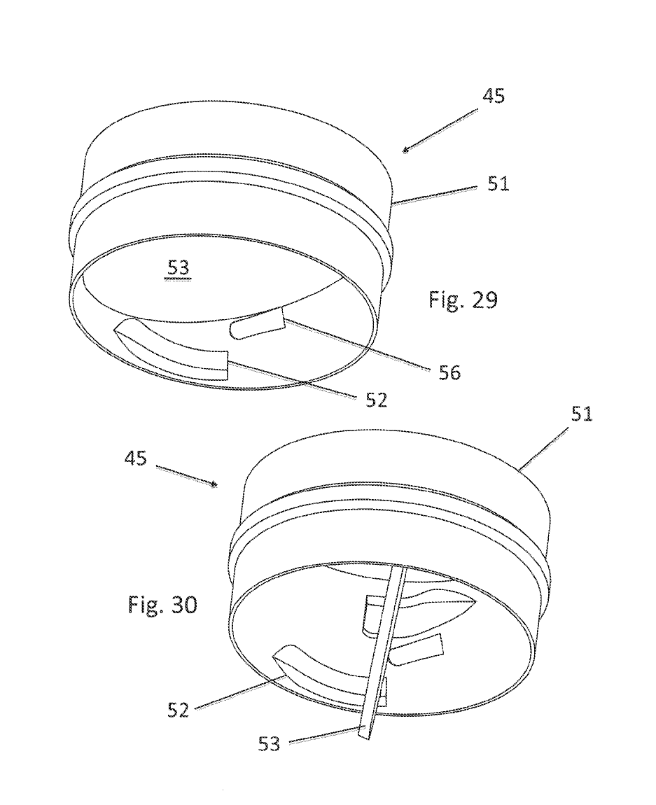

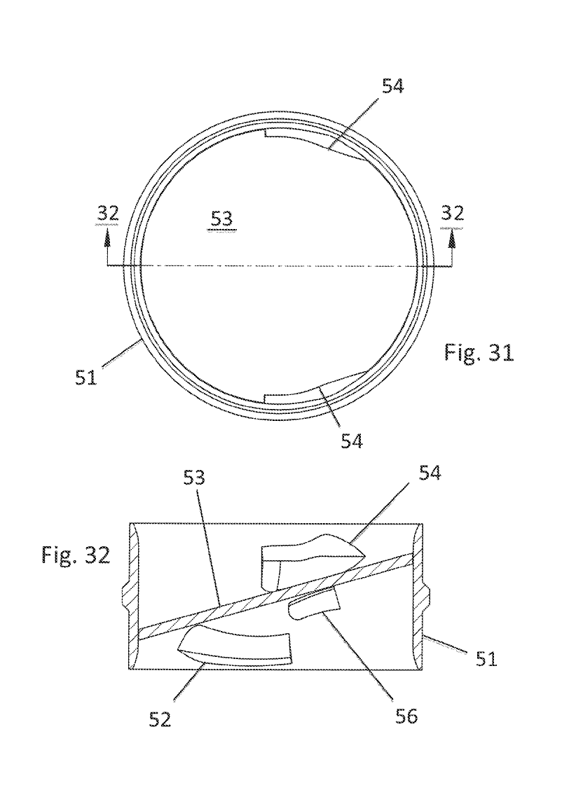

One alternative configuration of a valve 45 having a generally similar guiderail design is shown in FIGS. 27 through 31 which comprises a housing 51 and a flat plate occluder 53. Guiderails 52, 54 and 56 having similar curved and arcuate surfaces to those described hereinbefore and provide the continually shifting pivots axes between the occluder 53 and the housing 51. It can thus be seen that the invention can function with occluders with predominately flat faces as well as with an occluder with concave/convex surface features as previously described with respect to occluder 3. From the closed position shown in FIG. 32, it can be seen that the flat plate occluder 53 has a projected cylindrical perimeter that juxtaposes with the housing interior surface.

Although the invention has been described with regard to certain preferred embodiments which constitute the best mode known to the inventors at this time for carrying out their invention, it should be understood that various changes and modifications as would be obvious to one having ordinary skill in this art, may be made without departing from the scope of the invention which is described by the claims appended hereto. For example, although the preferred embodiment of the housing is described as having a right circular cylindrical midsection region, it should be understood that the other comparable cross sections can be used which would be satisfactory with an occluder of complementary shape.

1. A prosthetic mechanical heart valve which comprises:
a generally annular housing having a central passageway,
a single generally circular occluder shaped to close the central passageway through said housing,
a first pair of generally diametrically opposed guiderails which cooperate with said occluder in its closing pivotal movement,
a second pair of generally diametrically opposed guiderails which cooperate with said occluder in its opening pivotal movement, and
a third pair of diametrically opposed guiderails which cooperate with said occluder later during its opening pivotal movement to prevent said occluder from traveling downstream,
said first pair of guiderails having engaging surfaces which contact lateral regions of said occluder during its closing pivotal movement and create a pivot axis about which said occlude pivots to its closed position, which pivot axis shifts continuously so as to provide a quick and responsive closing pivotal movement during initial closing and then continuously slowing pivotal movement during the terminal closing cycle to minimize tangential velocity at the apexes of the occluder at final closing.

2. The heart valve of claim 1 wherein said second pair of guiderails have engaging surfaces which contact lateral regions of said occluder during its opening movement and create a pivot axis about which said occluder pivots to its fully open position, which pivot axis shifts continuously to provide a quick and responsive opening pivotal movement during an initial opening cycle segment and then slows opening pivotal movement during a terminal opening cycle segment to minimize occluder velocity at the end of the opening cycle.

3. The heart valve of claim 1 wherein said pivot axis is located where at least about 65% of the total occluder outflow face surface area acts to close the valve at the point of initial closing pivotal movement.

4. The heart valve of claim 3 wherein said pivot axis is located where the percentage of total occluder outflow face surface area where flow acts to close the valve changes continuously from at least about 75% at the point of initial closing pivotal movement to about 52% or less at full close.

5. The heart valve of claim 2 wherein said pivot axis is located where at least about 65% of the total occluder inflow face surface area acts to open the valve at the point of initial opening pivotal movement of the occluder.

6. The heart valve of claim 5 wherein said pivot axis is located where the percentage of total occluder inflow face surface area where flow acts to open the valve changes continuously from at least about 75% at the point of initial closing pivotal movement to no less than about 53% at full open.

7. The heart valve of claim 6 wherein said pivot axis is located where the percentage of total occluder inflow face surface area where flow acts to open the valve is at least about 60% at full open.

8. The heart valve of claim 1 wherein the occluder has a concave inflow face and a convex outflow face.

9. The heart valve of claim 8 wherein said occluder has a generally uniform thickness.

10. The heart valve of claim 9 wherein said occluder has a general circular periphery.

11. The heart valve of claim 1 wherein the occluder has a projected cylindrical perimeter and the housing has a midsection region that has an otherwise right circular cylindrical surface where said guiderails are located.

12. The heart valve of claim 11 wherein the occluder is a flat plate.

13. A prosthetic mechanical heart valve which comprises:
a generally annular housing having a central passageway having an axially center region with an interior surface of a right circular cylinder,
a single generally circular occluder positioned within said center region and shaped to close the central passageway through said housing, and
a plurality of pairs of generally diametrically opposed guiderails which protrude from said interior surface of said center region and cooperate with said occluder in its opening and closing pivotal movements,
said pairs of guiderails having a first set of engaging surfaces which contact lateral regions of said occluder during its closing pivotal movement and create a pivot axis about which said occluder pivots to its closed position, which pivot axis shifts continuously so as to provide a quick and responsive closing pivotal movement during initial closing and then continuously slowing pivotal movement during a terminal closing cycle to minimize tangential velocity at the apexes of the occluder at final closing.

14. The heart valve of claim 13 wherein said pairs of guiderails have a second set of engaging surfaces which contact lateral regions of said occluder during its opening movement and create a pivot axis about which said occluder pivots to its fully open position, which pivot axis shifts continuously to provide a quick and responsive opening pivotal movement during an initial opening cycle segment and then slows opening pivotal movement during a terminal opening cycle segment to minimize occluder velocity during final opening movement.
